(19) United States
(12) Patent Application Publication
(10) Pub. No.: US 2017/0252319 A1
(43) Pub. Date: Sep. 7, 2017

(54) Flavonoid-Poly(ethylene glycol) Complex Comprising Flavonoid Compounds and Poly(ethylene glycol) and Preparation Method Thereof

(71) Applicant: Korea Advanced Institute of Science and Technology, Daejeon (KR)

(72) Inventors: Haeshin Lee, Daejeon (KR); WhuiSu Shim, Daejeon (KR); Mihyun Lee, Daejeon (KR); Joseph Paul Park, Daejeon (KR)

(21) Appl. No.: 15/449,011

(22) Filed: Mar. 3, 2017

(30) Foreign Application Priority Data
Mar. 4, 2016 (KR) 10-2016-0026235

(51) Int. Cl. A61K 31/353; A61K 47/10

(52) U.S. Cl. CPC A61K 31/353; A61K 47/10

(57) ABSTRACT

The present invention relates to a flavonoid-poly(ethylene glycol) complex comprising flavonoid compounds and poly(ethylene glycol) and a preparation method of the same. The flavonoid-poly(ethylene glycol) complex of the invention displays a significantly improved solubility of flavonoid which is usually insoluble in an aqueous solution and contains flavonoid compounds distributed evenly in the complex so that the complex of the invention can be effectively used for the establishment of a method for enhancing solubility of flavonoid in an aqueous solution and a method for enhancing bioavailability of flavonoid.

### BACKGROUND OF THE INVENTION

The present invention relates to a flavonoid-poly(ethylene glycol) complex comprising flavonoid compounds and poly(ethylene glycol) and a preparation method of the same.

Flavonoid, a polyphenolic substance, is a natural substance that can be extracted from the nature. More than 5,000 flavonoid substances are distributed in various natural substances such plants as green tea and cacao, vegetables such as onion and parsley, and fruits such as grape-fruit and blueberry. The basic structure of flavonoid comprises three ring structures and is classified according to the chemical structure of the same. For example, the flavonoid compounds are classified into flavone, flavonol, flavanone, and flavan. The most representative flavonoid compounds are catechin, epigallocatechin gallate, epicatechin, quercetin, tannin, apigenin, myricetin, morin, resveratrol, luteolin, tangerit, and fisetin.

Flavonoid is a natural substance that is excellent in biocompatibility and displays various biochemical characteristics including anti-angiogenesis, anti-inflammation, anti-oxidant, anti-bacterial, and anti-viral activities, etc.

As described above, flavonoid has many favorable characteristics which are beneficial to human body, so that the natural flavonoid substances are expected to be used efficiently as an anti-cancer agent and an anti-inflammatory agent. In fact, among the flavonoid substances, epigallocatechin and apigenin have been confirmed to be effective in inhibiting angiogenesis and enhancing degenerative disease.

In the meantime, along with bioactivity, another important property of a drug is solubility. Only when a drug is dissolved in a solvent, it can be easily absorbed in human body and thereby the drug can be diffused in cells and also the concentration of a drug can be regulated. Water takes a major part of human body and therefore most of recent drugs are water-soluble.

However, most flavonoids have such a chemical structure that contains many hydrophobic ring structures, indicating that they are insoluble in an aqueous solution. Instead, they are soluble in an organic solvent, for example in methanol, ethanol, acetone, dimethyl sulfoxide (DMSO), chloroform, hexane, and acetic acid, etc. In particular, catechin displays high solubility in methanol, while quercetin is highly soluble in dimethyl sulfoxide. Myricetin is easily dissolved in ethanol. However, these organic solvents are toxic to human, so that they are limited in use for human body.

Even though flavonoids have many advantageous properties which are favorable for human, it is still hard to use flavonoids in the field of medicine due to the difficulty of regulating its absorption, diffusion, and concentration.

Thus, the present inventors tried to improve the insolubility of flavonoid in an aqueous solution so as to use it in a medicinal field. In the course of our study, the present inventors confirmed that a flavonoid-poly(ethylene glycol) complex comprising flavonoid compounds and poly(ethylene glycol) demonstrated an improved solubility in an aqueous solution, leading to the completion of the present invention.
A non-patent reference is Keumyeon Kim et al., AdvFunct Mater 2015, 25, 2402-2410

### SUMMARY OF THE INVENTION

It is an object of the present invention to provide a flavonoid-poly(ethylene glycol) complex comprising flavonoid compounds and poly(ethylene glycol).

It is another object of the present invention to provide a method for preparing the flavonoid-poly(ethylene glycol) complex above.

It is also an object of the present invention to provide a method for enhancing water-solubility of flavonoid comprising the step of preparing the flavonoid-poly(ethylene glycol) complex.

It is further an object of the present invention to provide a method for enhancing bioavailability of flavonoid comprising the step of preparing the flavonoid-poly(ethylene glycol) complex.

To achieve the above objects, the present invention provides a flavonoid-poly(ethylene glycol) complex comprising flavonoid compounds and poly(ethylene glycol).

The present invention also provides a method for preparing the flavonoid-poly(ethylene glycol) complex comprising the following steps:

preparing a mixed solution by adding flavonoid compounds into a poly(ethylene glycol) aqueous solution (step 1);

drying the mixed solution prepared in step 1 (step 2).

Further, the present invention provides a method for enhancing water-solubility of flavonoid comprising the step of preparing the flavonoid-poly(ethylene glycol) complex.

Also, the present invention provides a method for enhancing bioavailability of flavonoid comprising the step of preparing the flavonoid-poly(ethylene glycol) complex.

The flavonoid-poly(ethylene glycol) complex of the present invention displays a significantly improved solubility of flavonoid which is usually insoluble in an aqueous solution and contains flavonoid compounds distributed evenly in the complex so that the complex of the invention can be effectively used for the establishment of a method for enhancing solubility of flavonoid in an aqueous solution and a method for enhancing bioavailability of flavonoid.

### BRIEF DESCRIPTION OF THE DRAWINGS

The application of the preferred embodiments of the present invention is best understood with reference to the accompanying drawings, wherein:

FIG. 1 is a photograph showing the solution in which the non-treated catechin (insoluble catechin) and the catechin-poly(ethylene glycol) (PEG) complex obtained by converting the insoluble catechin into the soluble catechin with regulating the weight part of catechin and poly(ethylene glycol) (PEG) prepared in Example 1 are dissolved.

FIG. 2 is a photograph showing the solution in which the non-treated morin (insoluble morin) and the morin-poly(ethylene glycol) (PEG) complex obtained by converting the insoluble morin into the soluble morin with regulating the weight part of morin and poly(ethylene glycol) (PEG) prepared in Example 2 are dissolved.

FIG. 3 is a photograph showing the solution in which the non-treated quercetin (insoluble quercetin) and the quercetin-poly(ethylene glycol) (PEG) complex obtained by converting the insoluble quercetin into the soluble quercetin with regulating the weight part of quercetin and poly(ethylene glycol) (PEG) prepared in Example 3 are dissolved.

FIG. 4 is a graph illustrating the solubility of the insoluble catechin and the catechin-PEG complex of Example 1, analyzed by UV/VIS spectroscopy.

FIG. 5 is an image illustrating the formation of the catechin-PEG complex, analyzed by TEM.

FIG. 6 is a graph illustrating the particle size of the catechin-PEG complex.

FIG. 7 is a chart illustrating the concentration-dependent quantification of dissolved catechin.

### DESCRIPTION OF THE PREFERRED EMBODIMENTS

Hereinafter, the present invention is described in detail.

The present invention provides a flavonoid-poly(ethylene glycol) complex comprising flavonoid compounds and poly(ethylene glycol).

Hereinafter, the flavonoid-poly(ethylene glycol) complex is described in more detail.

Flavonoid has beneficiary characteristics to living bodies, so that it is expected to have high efficacy when used as medicines for anticancer therapy and inflammation treatment. However, due to the chemical structure that contains many hydrophobic rings, flavonoid is insoluble in an aqueous solution, which makes it difficult to use in the human body. In addition, in order to maintain the unique efficacy of the flavonoid, it is important to increase the solubility in water without chemical modification of the flavonoid.

The flavonoid-poly(ethylene glycol) complex of the present invention is the complex in which flavonoid and poly(ethylene glycol) are bound by hydrogen bonding.

Since flavonoid and poly(ethylene glycol) are bound by physical conjugation using hydrogen bonding instead of chemical bonding, the chemical structure of the flavonoid is not changed and the solubility in water is increased. Thus, the complex is easy to use in living body while maintaining the unique pharmaceutical efficacy.

The said flavonoid is selected from the group consisting of catechin, morin, quercetin, epigallocatechin gallate, epicatechin, tannin, apigenin, myricetin, resveratrol, luteolin, tangerit, fisetin, flavanone, kaempferol, hesperetin, and naringenin, but not always limited thereto and more preferably it is selected from the group consisting of catechin, morin, and quercetin.

Further, the number average molecular weight of PEG above is preferably 4000˜20,000 g/mol, but not always limited thereto.

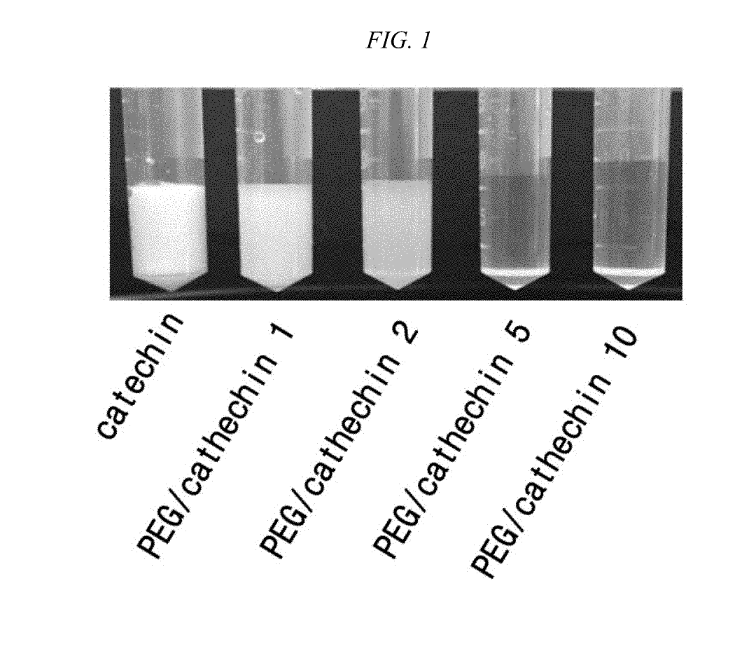

The solubility of the flavonoid-poly(ethylene glycol) complex of the invention was investigated. As a result, it was confirmed that the complex of the invention had excellent water-solubility, compared with the non-treated flavonoid (insoluble flavonoid). In particular, as the ratio of poly(ethylene glycol) in the flavonoid-poly(ethylene glycol) complex increased, the solubility also increased significantly (see step 2 of Examples 1, 2 and 3, and FIGS. 1, 2, and 3).

Since flavonoid and poly(ethylene glycol) are bound by physical conjugation using hydrogen bonding instead of chemical bonding, the chemical structure of the flavonoid is not changed and the solubility in water is increased. Thus, the complex is easy to use in living body while maintaining the unique pharmaceutical efficacy.

The present invention also provides a method for preparing the flavonoid-poly(ethylene glycol) complex comprising the following steps:

preparing a mixed solution by adding flavonoid compounds into a poly(ethylene glycol) aqueous solution (step 1);

drying the mixed solution prepared in step 1 (step 2).

Hereinafter, the method for preparing the flavonoid-poly(ethylene glycol) complex is described in more detail.

In the method for preparing the flavonoid-poly(ethylene glycol) complex of the invention, step 1 is to prepare a mixed solution by adding flavonoid into a poly(ethylene glycol) aqueous solution.

At this time, the flavonoid can be added as dissolved in an organic solvent. The organic solvent herein can be a water-miscible organic solvent, but not always limited thereto.

Preferably, a polar protic solvent such as methanol, ethanol, propanol, isopropanol, butanol, and acetic acid or a polar aprotic solvent such as dimethylformamide (DMF), dimethylsulfoxide (DMSO), tetrahydrofuran (THF), and acetone can be selected, and more preferably methanol, ethanol, acetone, or DMSO can be selected.

In the method for preparing the flavonoid-poly(ethylene glycol) complex of the invention, step 2 is to dry the mixed solution obtained in step 1.

Step 2 can be performed by the following steps but not always limited thereto:

eliminating the organic solvent included in the mixed solution obtained in step 1 (step a); and

freeze-drying the organic solvent-free mixed solution obtained in step a (step b).

The flavonoid-poly(ethylene glycol) complex prepared by the method for preparing a flavonoid-poly(ethylene glycol) complex of the present invention is the complex conjugated by hydrogen bonding, so that there is no change in the chemical structure thereof.

The solubility of the flavonoid-poly(ethylene glycol) complex prepared by the method for preparing a flavonoid-poly(ethylene glycol) complex of the present invention was investigated. As a result, compared with the non-treated flavonoid (insoluble flavonoid), the solubility of the flavonoid-poly(ethylene glycol) complex of the invention was increased (see step 2 of Examples 1, 2 and 3, and FIGS. 1, 2, and 3).

Therefore, it was confirmed that the water-solubility of insoluble flavonoid can be increased by the method for preparing a flavonoid-poly(ethylene glycol) complex of the invention without any chemical change. So, the method of the invention above can be effectively used for increasing the solubility of the insoluble flavonoid.

The present invention also provides a method for enhancing water-solubility of flavonoid comprising the step of preparing the flavonoid-poly(ethylene glycol) complex.

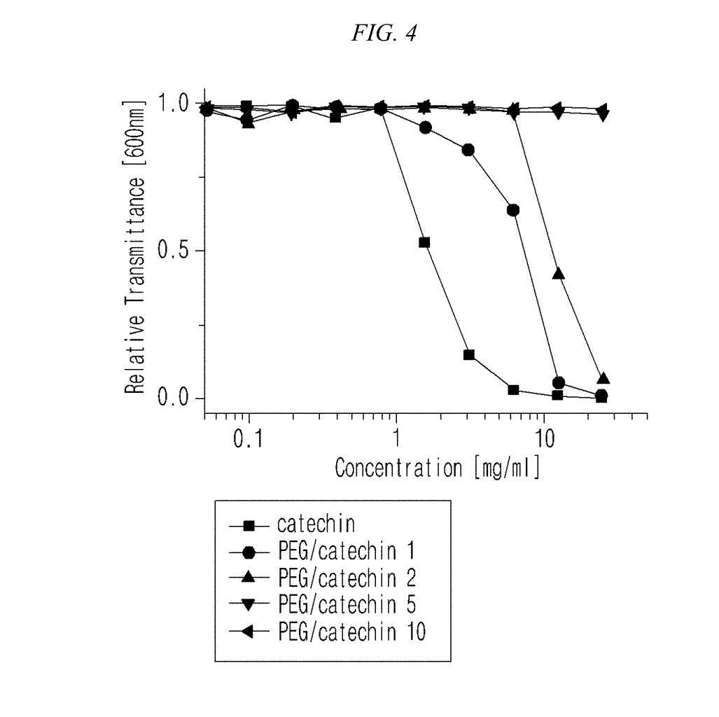

The increase of the solubility of flavonoid in an aqueous solution according to the method for enhancing water-solubility of flavonoid comprising the step of preparing the flavonoid-poly(ethylene glycol) complex was investigated. As a result, the flavonoid-poly(ethylene glycol) complex produced by the method above displayed an excellent permeability, compared with the insoluble flavonoid, so that it can be suggested that the solubility was increased. When it was dissolved at a high concentration, the permeability was confirmed as 1, indicating that the solubility was significantly increased (see Experimental Example 1 and FIG. 4).

Therefore, the method for enhancing water-solubility of flavonoid comprising the step of preparing the flavonoid-poly(ethylene glycol) complex was confirmed to be efficient in increasing the solubility of the insoluble flavonoid.

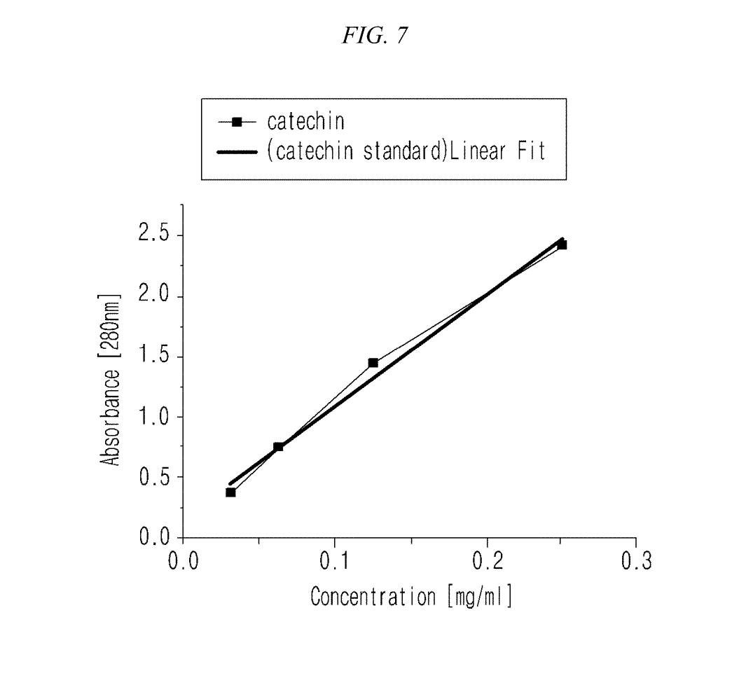

The uniformity of the flavonoid-poly(ethylene glycol) complex produced by the method for enhancing water-solubility of flavonoid comprising the step of preparing the flavonoid-poly(ethylene glycol) complex was investigated. As a result, the flavonoid-poly(ethylene glycol) complex produced by the method for enhancing water-solubility of flavonoid showed consistent concentration and absorbance in all the randomly selected samples, indicating that the flavonoid was dispersed evenly in the complex (see Experimental Example 3, FIG. 7, and Table 1).

Therefore, it was confirmed that the method for enhancing water-solubility of flavonoid comprising the step of preparing the flavonoid-poly(ethylene glycol) complex of the invention is efficient in increasing the solubility of the insoluble flavonoid and at the same time in even distribution of flavonoid having a unique pharmaceutical efficacy in the flavonoid-poly(ethylene glycol) complex, so that the method can be used to increase the water-solubility of flavonoid.

In addition, the present invention provides a method for enhancing bioavailability of flavonoid comprising the step of preparing the flavonoid-poly(ethylene glycol) complex.

The method for enhancing bioavailability of flavonoid comprising the step of preparing the flavonoid-poly(ethylene glycol) complex of the invention is efficient in increasing the solubility of the flavonoid-PEG complex in an aqueous solution with allowing the flavonoid to maintain its unique pharmaceutical efficacy, and also helps the absorption in living body, which favors brining the pharmaceutical effect.

Practical and presently preferred embodiments of the present invention are illustrative as shown in the following Examples.

However, it will be appreciated that those skilled in the art, on consideration of this disclosure, may make modifications and improvements within the spirit and scope of the present invention.

### Example 1

### Method for Enhancing Water-Solubility of Catechin

### Step 1

### Preparation of Catechin-PEG Complex

Catechin hydrate (MW=290.27 g/mol, Sigma Aldrich) was dissolved in an organic solvent (methanol, ethanol, acetone, DMSO, etc) at the concentration of 10 mg/ml. Poly(ethylene glycol) (PEG, MW=4 k, 8 k, 10 k, and 20 kg/mol, Sigma Aldrich) was dissolved in distilled water at the concentration of 2, 4, 10, and 20 mg/ml, respectively. The prepared two solutions were mixed together (catechin:PEG=1:5 (v/v)), followed by mixing vigorously. The mixed solution was loaded in a high-temperature evaporator (Centrifan™ PE KDScientific), followed by evaporation of the organic solvent at 50° C. for 2 hours. The remaining mixture was frozen rapidly in liquid nitrogen. The frozen mixture was dried by freeze-drying for 2˜3 days. As a result, the dried catechin-PEG complex was prepared.

### Step 2

### Confirmation of Water-Solubility of Catechin

To investigate the water-solubility of the dried catechin-PEG complex prepared in step 1 above, the dried catechin-PEG complex powder was dissolved in an aqueous solution solvent such as saline or biological buffer at a high concentration, followed by confirmation of the solubility.

FIG. 1 is a photograph showing the solution in which the non-treated catechin (insoluble catechin) and the catechin-poly(ethylene glycol) (PEG) complex obtained by converting the insoluble catechin into the soluble catechin with regulating the weight part of catechin and poly(ethylene glycol) (PEG) prepared in Example 1 are dissolved.

As shown in FIG. 1, the solution of the non-treated catechin is an opaque solution which showed very low solubility in water. In the meantime, the solution containing the catechin-PEG complex of Example 1 of the invention wherein catechin was mixed with PEG at the ratio of 1:1, 2, 5, and 10 (weight part) showed higher solubility in water. In particular, when PEG was mixed with catechin at the ratio of 5:1 or higher (weight part), the solution turned into transparent, indicating the mixture was completely dissolved.

### Example 2

### Method for Enhancing Water-Solubility of Morin

### Step 1

### Preparation of Morin-PEG Complex

Morin hydrate (Sigma Aldrich) was dissolved in an organic solvent (methanol, ethanol, acetone, DMSO, etc) at the concentration of 10 mg/ml. Poly(ethylene glycol) (PEG, MW=4 k, 8 k, 10 k, and 20 kg/mol, Sigma Aldrich) was dissolved in distilled water at the concentration of 20, 100, 200, 500, and 1000 mg/ml, respectively. The prepared two solutions were mixed together (morin:PEG=1:5 (v/v)), followed by mixing vigorously for 10 minutes. The mixed solution was loaded in a high-temperature evaporator (Centrifan™ PE KDScientific), followed by evaporation of the organic solvent at 50° C. for 2 hours. The remaining mixture was frozen rapidly in liquid nitrogen. The frozen mixture was dried by freeze-drying for 2˜3 days. As a result, the dried morin-PEG complex was prepared.

### Step 2

### Confirmation of Water-Solubility of Morin

To investigate the water-solubility of the dried morin-PEG complex prepared in step 1 above, the dried morin-PEG complex powder was dissolved in an aqueous solution solvent such as saline or biological buffer at a high concentration, followed by confirmation of the solubility.

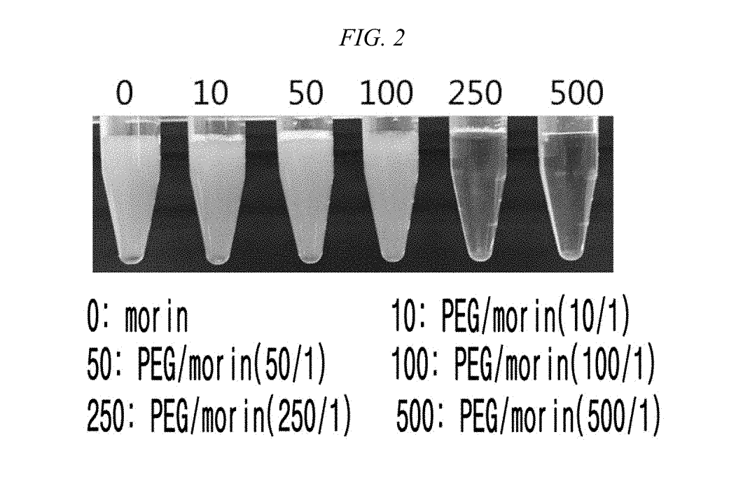

FIG. 2 is a photograph showing the solution in which the non-treated morin (insoluble morin) and the morin-poly(ethylene glycol) (PEG) complex obtained by converting the insoluble morin into the soluble morin with regulating the weight part of morin and poly(ethylene glycol) (PEG) prepared in Example 2 are dissolved.

As shown in FIG. 2, the solution of the non-treated morin is an opaque solution which showed very low solubility in water. In the meantime, the solution containing the morin-PEG complex of Example 2 of the invention wherein morin was mixed with PEG at the ratio of 1:10, 50, 100, 250, and 500 (weight part) showed higher solubility in water. In particular, when PEG was mixed with morin at the ratio of 250:1 or higher (weight part), the solution turned into transparent, indicating the mixture was completely dissolved.

### Example 3

### Method for Enhancing Water-Solubility of Quercetin

### Step 1

### Preparation of Quercetin-PEG Complex

Quercetin hydrate (Sigma Aldrich) was dissolved in an organic solvent (methanol, ethanol, acetone, DMSO, etc) at the concentration of 10 mg/ml. Poly(ethylene glycol) (PEG, MW=4 k, 8 k, 10 k, and 20 kg/mol, Sigma Aldrich) was dissolved in distilled water at the concentration of 10, 50, 100, 500, and 1000 mg/ml, respectively. The prepared two solutions were mixed together (quercetin:PEG=1:5 (v/v)), followed by mixing vigorously for 10 minutes. The mixed solution was loaded in a high-temperature evaporator (Centrifan™ PE KDScientific), followed by evaporation of the organic solvent at 50° C. for 2 hours. The remaining mixture was frozen rapidly in liquid nitrogen. The frozen mixture was dried by freeze-drying for 2˜3 days. As a result, the dried quercetin-PEG complex was prepared.

### Step 2

### Confirmation of Water-Solubility of Quercetin

To investigate the water-solubility of the dried quercetin-PEG complex prepared in step 1 above, the dried quercetin-PEG complex powder was dissolved in an aqueous solution solvent such as saline or biological buffer at a high concentration, followed by confirmation of the solubility.

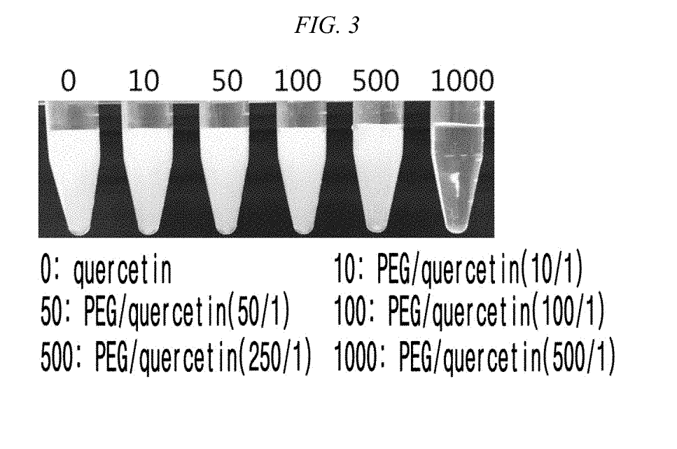

FIG. 3 is a photograph showing the solution in which the non-treated quercetin (insoluble quercetin) and the quercetin-poly(ethylene glycol) (PEG) complex obtained by converting the insoluble quercetin into the soluble quercetin with regulating the weight part of quercetin and poly(ethylene glycol) (PEG) prepared in Example 2 are dissolved.

As shown in FIG. 3, the solution of the non-treated quercetin is an opaque solution which showed very low solubility in water. In the meantime, the solution containing the quercetin-PEG complex of Example 3 of the invention wherein quercetin was mixed with PEG at the ratio of 1:10, 50, 100, 500, and 1000 (weight part) showed higher solubility in water. In particular, when PEG was mixed with quercetin at the ratio of 250:1 or higher (weight part), the solution turned into transparent, indicating the mixture was completely dissolved.

### Experimental Example 1

### Evaluation of Increase of Water-Solubility of Catechin

To evaluate the increase of water-solubility of the catechin-PEG complex obtained in Example 1 of the invention, UV/VIS spectroscopy (8453 Hewlett Packard) was performed and the results are shown in FIG. 4.

Particularly, the transparency of a solution in which the catechin-PEG complex was dissolved was measured and presented as the permeability at 600 nm.

FIG. 4 is a graph illustrating the solubility of the insoluble catechin and the catechin-PEG complex of Example 1, analyzed by UV/VIS spectroscopy.

As shown in FIG. 4, the catechin-PEG complex of the invention displayed the higher permeability than that of the insoluble catechin, indicating that the solubility of the complex was increased. In particular, the permeability of the solution containing high concentrations of the catechin-PEG (catechin/PEG=⅕ or catechin/PEG= 1/10, weight part) was 1, indicating that the solubility was significantly increased.

Therefore, it was confirmed that the method for enhancing water-solubility of insoluble flavonoids using poly(ethylene glycol) is efficient in increasing solubility of catechin which is generally insoluble in an aqueous solution.

### Experimental Example 2

### Confirmation of Catechin-PEG Complex Formation

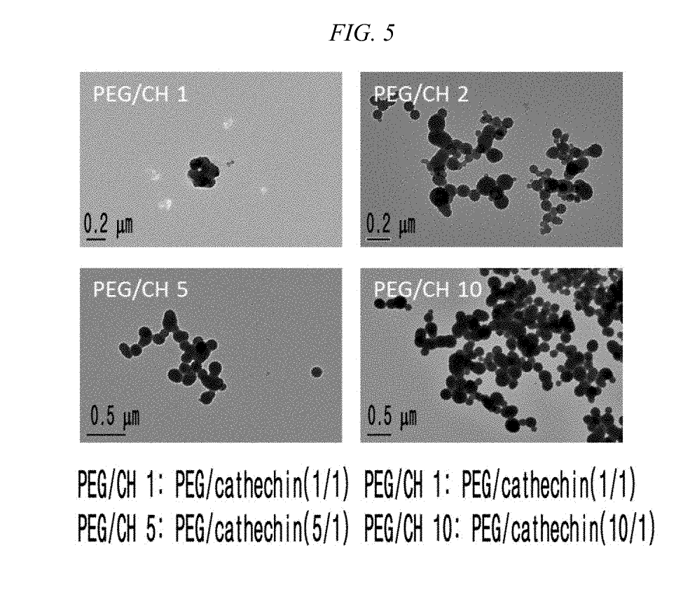

To confirm the formation of the catechin-PEG complex obtained in Example 1, TEM (Transmission Electron Microscopy) assay was performed and the results are shown in FIG. 5.

FIG. 5 is an image illustrating the formation of the catechin-PEG complex, analyzed by TEM.

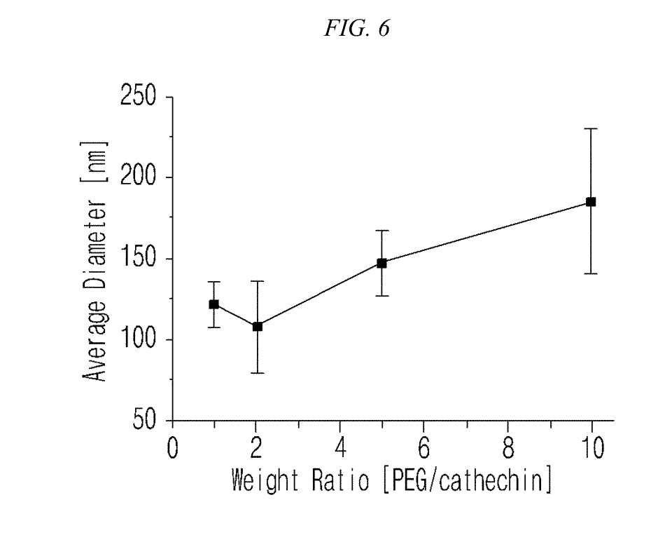

As shown in FIG. 5, the catechin-PEG complex was formed as spherical particles. The size of the particles observed in FIG. 5 was measured and the results are shown in FIG. 6.

FIG. 6 is a graph illustrating the particle size of the catechin-PEG complex.

As shown in FIG. 6, the catechin-PEG complex obtained in Example 1 was formed as spherical particles in the mean size of 100˜200 nm.

### Experimental Example 3

### Evaluation of Uniformity of Soluble Catechin-PEG Complex

The uniformity of the catechin-PEG complex prepared in Example 1 was investigated. The catechin quantification curve (standard curve) obtained by measuring the signal intensity at 280 nm (UV) at which the catechin absorption takes place by the concentration range of 0.01˜0.5 mg/ml is shown in FIG. 7.

FIG. 7 is a chart illustrating the concentration-dependent quantification of dissolved catechin.

Five samples were taken randomly from the catechin-PEG complex powder and dissolved at the concentration of 0.6 mg/ml. The catechin concentration calculated with the weight ratio of catechin to PEG was 0.1 mg/ml. The comparative catechin concentration obtained by UV/VIS spectroscopy and comparison with the standard curve above is shown in Table 1 below.

TABLE 1

| | | UV | |
|---|---|---|---|
| Catechin-PEG | | absorbance | Measured conc. |
| complex | Conc. (n = 5) | [280 nm] | [mg/ml] |
| catechin 1 | 0.1 mg/ml | 1.1960 | 0.11203 |
| weight | | | |
| part/PEG 5 | | | |
| weight part | | | |
| | 0.1 mg/ml | 1.0130 | 0.0922532 |
| | 0.1 mg/ml | 1.1536 | 0.10744 |
| | 0.1 mg/ml | 1.1401 | 0.10598 |
| | 0.1 mg/ml | 1.0935 | 0.10095 |

| | |
|---|---|
| Average | 0.10373 ± 0.0067 |
| | mg/ml |

Table 1 presents the homogeneity distribution of the catechin-PEG complex powder prepared by using PEG and catechin at the weight ratio of 5:1.

As shown in Table 1, the catechin-PEG complex prepared by the method of the invention to increase water-solubility of the insoluble flavonoids using poly(ethylene glycol) displayed consistent concentration and absorbance in all of those 5 samples, indicating that catechin was evenly distributed in the complex.

Therefore, it was confirmed that the unique characteristics of catechin was evenly distributed in the catechin-PEG physical complex.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended Claims.

1. A flavonoid-poly(ethylene glycol) complex comprising flavonoid compounds and poly(ethylene glycol).

2. The flavonoid-poly(ethylene glycol) complex according to claim 1, wherein the flavonoid and the poly(ethylene glycol) are conjugated by hydrogen bonding.

3. The flavonoid-poly(ethylene glycol) complex according to claim 1, wherein the flavonoid is selected from the group consisting of catechin, morin, quercetin, epigallocatechin gallate, epicatechin, tannin, apigenin, myricetin, resveratrol, luteolin, tangerit, and fisetin.

4. The flavonoid-poly(ethylene glycol) complex according to claim 1, wherein the number average molecular weight of the PEG is 4000˜20,000 g/mol.

5. A method for preparing the flavonoid-poly(ethylene glycol) complex of claim 1 comprising the following steps:
preparing a mixed solution by adding flavonoid compounds into a poly(ethylene glycol) aqueous solution (step 1);
drying the mixed solution prepared in step 1 (step 2).

6. The method for preparing the flavonoid-poly(ethylene glycol) complex according to claim 5, wherein the flavonoid is added as dissolved in an organic solvent.

7. The method for preparing the flavonoid-PEG complex according to claim 6, wherein the organic solvent is a water-miscible organic solvent.

8. A method for enhancing water-solubility of flavonoid comprising the step of preparing the flavonoid-poly(ethylene glycol) complex of claim 1.

9. A method for enhancing bioavailability of flavonoid comprising the step of preparing the flavonoid-poly(ethylene glycol) complex of claim 1.

10. The method according to claim 9, wherein the flavonoid and the poly(ethylene glycol) are conjugated by hydrogen bonding.

11. The method according to claim 9, wherein the flavonoid is selected from the group consisting of catechin, morin, quercetin, epigallocatechin gallate, epicatechin, tannin, apigenin, myricetin, resveratrol, luteolin, tangerit, and fisetin.

12. The method according to claim 9, wherein the number average molecular weight of the PEG is 4000˜20,000 g/mol.

13. The method according to claim 5, wherein the flavonoid and the poly(ethylene glycol) are conjugated by hydrogen bonding.

14. The method according to claim 5, wherein the flavonoid is selected from the group consisting of catechin, morin, quercetin, epigallocatechin gallate, epicatechin, tannin, apigenin, myricetin, resveratrol, luteolin, tangerit, and fisetin.

15. The method according to claim 5, wherein the number average molecular weight of the PEG is 4000˜20,000 g/mol.
